(19) United States
(12) Patent Application Publication
(10) Pub. No.: US 2008/0140081 A1
(43) Pub. Date: Jun. 12, 2008

(54) CUT GUIDES

(75) Inventors: Jackson R. Heavener, Warsaw, IN (US); Bradley Stout, Fort Wayne, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 11/949,357

(22) Filed: Dec. 3, 2007

Related U.S. Application Data
(60) Provisional application No. 60/868,469, filed on Dec. 4, 2006.

(51) Int. Cl. A61B 17/58

(52) U.S. Cl. 606/87

(57) ABSTRACT

An orthopedic cut guide including a motor configured to actuate a cutter guide having a guide surface. In one exemplary embodiment, the cut guide may include a plurality of motors each configured to actuate the cutter guide. Advantageously, by utilizing a combination of motors, the cutter guide and, correspondingly, the guide surface may be actuated along multiple planes and/or in multiple directions to position the cut guide in the desired position to facilitate resection of a bone.

### CROSS-REFERENCE TO RELATED APPLICATIONS

This application claims the benefit under Title 35 U.S.C. § 119(e) of U.S. Provisional Patent Application Ser. No. 60/868,469, entitled CUT GUIDES, filed on Dec. 4, 2006, the entire disclosure of which is expressly incorporated by reference herein.

### BACKGROUND

1. Field of the Invention

The present invention relates to orthopedic devices, and, particularly, to orthopedic cut guides.

2. Description of the Related Art

A number of orthopedic procedures require the resection of a patient's natural bone stock. For example, if the bone stock is diseased or damaged, that portion of the bone stock may be resected. In order to remove the diseased or damaged bone stock, it may be necessary for the surgeon to make several different cuts and may also result in the removal of a significant portion of the bone. However, to provide an attachment surface or a supportive base for an orthopedic implant, the surgeon may align the cuts in a manner that allows for the retention of as much of the healthy bone stock as possible.

To assist the surgeon in aligning the cuts, a cut guide may be used. A cut guide may provide a guide surface for supporting or receiving a cutting tool that is manipulated by the surgeon. For example, the guide surface may be a flat surface along which the surgeon guides the cutting tool during the resection. Additionally, cut guides may include a plurality of different guide surfaces to allow the surgeon to use a single device to guide multiple cuts. Alternatively, the cut guide may be manipulated by the surgeon to reposition the cut guide after a first cut to place the cut guide in the desired position for a second cut.

### SUMMARY

The present invention relates to orthopedic devices, and, particularly, to orthopedic cut guides. In one exemplary embodiment, the orthopedic cut guide of the present invention includes a motor configured to actuate a cutter guide having a guide surface. In another exemplary embodiment, the cut guide may include a plurality of motors each configured to actuate the cutter guide. Advantageously, by utilizing a combination of motors, the cutter guide and, correspondingly, the guide surface may be actuated along multiple planes and/or in multiple directions to position the cut guide in the desired position to facilitate resection of a bone.

In one exemplary embodiment, the cut guide includes a housing containing at least one motor therein and a bone mount connected to the housing. The bone mount is configured to be attached to the bone to mount the cut guide adjacent the bone. For example, the bone mount may include a plurality of apertures configured to receive fasteners therethrough. Additionally, the at least one motor may be operably connected to an adjustment arm, which is in turn connected to the cutter guide. Thus, during operation of the at least one motor, the adjustment arm is actuated causing corresponding actuation of the cutter guide.

In another exemplary embodiment, each motor of the present cut guide is electronically connected to a position sensor. The position sensor monitors the operation of the motor to determine the position of the motor. The position sensor may also be electronically connected to a motor control unit that receives the position information from the position sensor and calculates the resulting position of the cutter guide. The motor control unit may also supply power to the motors to move the cutter guide into the position requested by a user. In one exemplary embodiment, the user may request movement of the cut guide through a user interface unit that is electronically connected to the motor control unit. Once the user input is sent by the user interface unit to the motor control unit, the motor control unit may then activate the motors to move the cutter guide into the requested position. In order to ensure that the cut guide is in the proper position, the motor control unit monitors feedback received from the position sensors and operates the motors accordingly.

In one form thereof, the present invention provides a cut guide for the resection of a bone, the cut guide including: a housing; a motor contained within said housing; a bone mount connected to said housing; at least one adjustment arm received by said housing and operably connected to said motor, whereby operation of said motor results in movement of said at least one adjustment arm; and a cutter guide having a cut surface for guiding a cutting tool, said cutter guide attached to said at least one adjustment arm, whereby movement of said at least one adjustment arm results in corresponding movement of said cutter guide.

In another form thereof, the present invention provides a cut guide for the resection of a bone, the cut guide including: a housing; a bone mount connected to said housing; a cutter guide having a cut surface for guiding a cutting tool; and means for automatically adjusting the orientation of the guide surface of the cutter guide relative to the housing in response to a user input.

In yet another form thereof, the present invention provides a method of resecting a bone, including: providing a cutter guide having a guide surface for guiding a cutting tool; moveably connecting the cutter guide to a bone mount; mounting the cutter guide to the bone with the bone mount; providing a user input indicating a desired position of the cutter guide relative to the bone mount and, therefore, the bone; and automatically actuating the cutter guide to the desired position.

### BRIEF DESCRIPTION OF THE DRAWINGS

The above-mentioned and other features and advantages of this invention, and the manner of attaining them, will become more apparent and the invention itself will be better understood by reference to the following description of an embodiment of the invention taken in conjunction with the accompanying drawings, wherein:

FIG. 1 is a perspective view of a cut guide positioned on the anterior side of a femur according to one embodiment of the present invention;

FIG. 2 is a side view of the device of FIG. 1 taken along line 2-2 of FIG. 1;

FIG. 3 is a distal view of the device of FIG. 2 taken along line 3-3 of FIG. 2;

FIG. 4 is an anterior view of the device of FIG. 2 taken along line 4-4 of FIG. 2;

FIG. 5 is a cross-sectional view of the device of FIG. 2 taken along line 5-5 of FIG. 2;

FIG. 6 is a side view of the device of FIG. 1 taken from the perspective of line 2-2 of FIG. 1 and depicting the cutter guide at a neutral flexion/extension or anterior/posterior slope angle;

FIG. 7 is a side view of the device of FIG. 1 taken from the perspective of line 2-2 of FIG. 1 and depicting the cutter guide at a first flexion/extension or anterior/posterior slope angle;

FIG. 8 is a side view of the device of FIG. 1 taken from the perspective of line 2-2 of FIG. 1 and depicting the cutter guide at a second flexion/extension or anterior/posterior slope angle;

FIG. 9 is an anterior view of the device of FIG. 1 taken from the perspective of line 4-4 of FIG. 2 and depicting the cutter guide at a neutral varus-valgus angle;

FIG. 10 is an anterior view of the device of FIG. 1 taken from the perspective of line 4-4 of FIG. 2 and depicting the cutter guide at a first varus-valgus angle;

FIG. 10 is an anterior view of the device of FIG. 1 taken from the perspective of line 4-4 of FIG. 2 and depicting the cutter guide at a second varus-valgus angle; and

FIG. 12 is a schematic diagram of a cut guide control system for operation of the cut guide of FIG. 1.

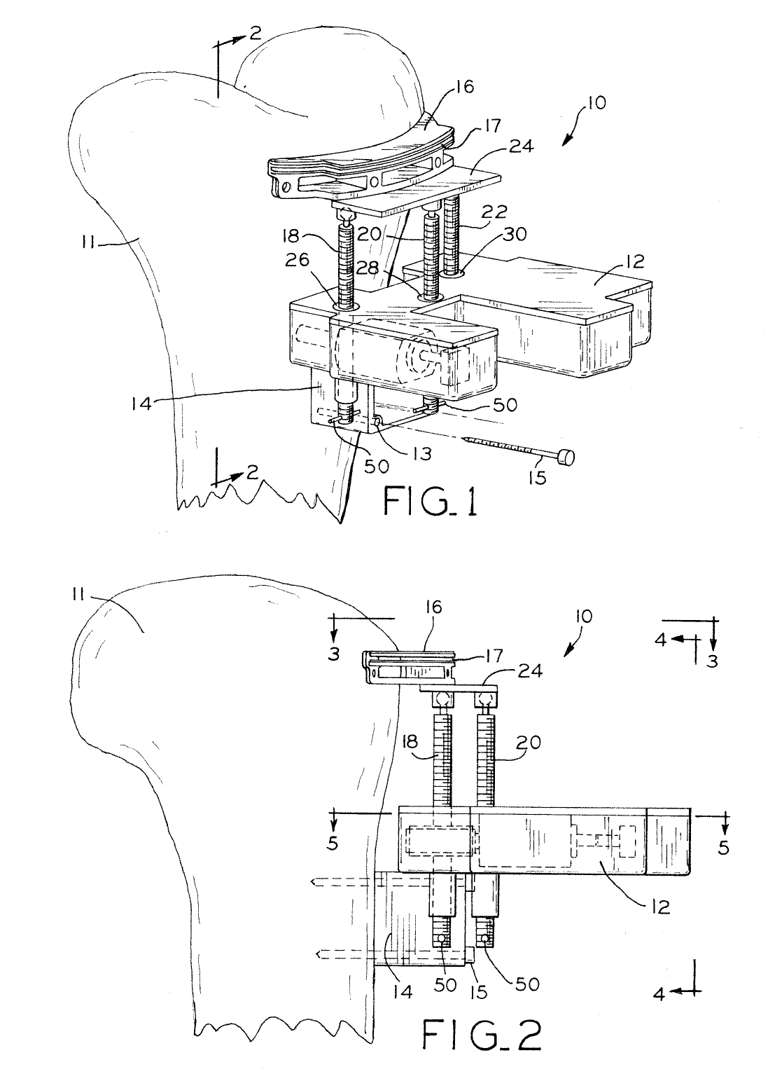

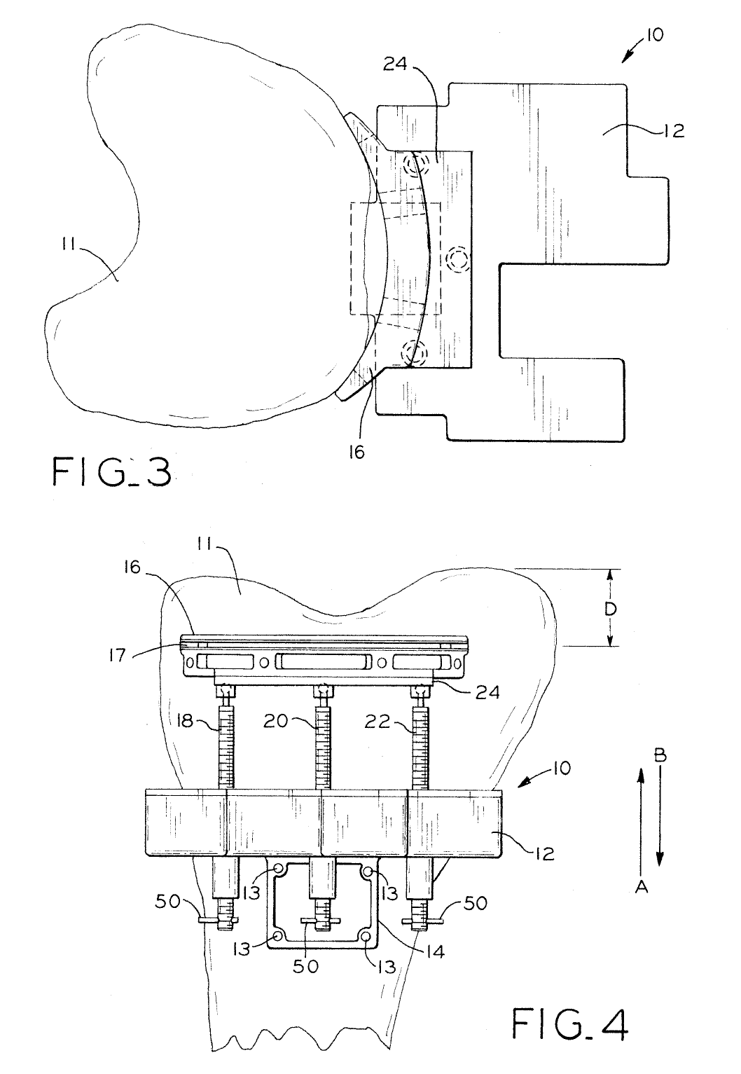

Corresponding reference characters indicate corresponding parts throughout the several views. The exemplification set out herein illustrates one preferred embodiment of the invention, in one form, and such exemplification is not to be construed as limiting the scope of the invention any manner.

### DETAILED DESCRIPTION

Referring to FIG. 1, cut guide 10 is shown including housing 12, bone mount 14, and cutter guide 16. Bone mount 14 may be positioned against a bone, such as femur 11, and may include apertures 13 sized to receive fasteners, such as pins 15, therethrough. By passing pins 15 through apertures 13 and into femur 11, bone mount 14 secures cut guide 10 to femur 11. Alternatively, bone mount 14 may be formed for attachment in a manner described in U.S. patent application Ser. No. 11/623,489, entitled ORTHOPEDIC DEVICE FOR SECURING TO TISSUE, filed on Jan. 16, 2007, the entire disclosure of which is expressly incorporated by reference herein.

Cutter guide 16 includes a cut surface, in the form of cut slot 17, sized to receive and guide a cutting tool during resection of femur 11. While described and depicted as a cut slot, the cut surface of the present invention may be any planar surface capable of guiding a cutting tool during bone resection. Connecting cutter guide 16 to housing 12 are adjustment arms 18, 20, 22. In one exemplary embodiment, adjustment arms 18, 20, 22 are formed as threaded pins. Further, adjustment arms 18, 20, 22 each define a longitudinal axis and include a substantially spherical ball head, shown in dashed lines in FIGS. 9-11, which are received within corresponding substantially spherical sockets on bracket 24 of cutter guide 16 to form a constrained ball-and-socket connection. Specifically, a constrained ball-and-socket connection is formed when the socket contacts the ball over an area larger than a hemispherical area of the ball, i.e., the socket extends more than halfway around the ball. As a result, the ball is allowed to rotate in the socket along three orthogonal axes, but is prevented from separating from, e.g., translating in a direction away from, the socket. Thus, by utilizing a constrained ball-and-socket connection, cutter guide 16 remains securely connected to each of the ball heads of adjustment arms 18, 20, 22, while retaining the ability to independently move about the ball heads of each of adjustment arms 18, 20, 22 along multiple axes.

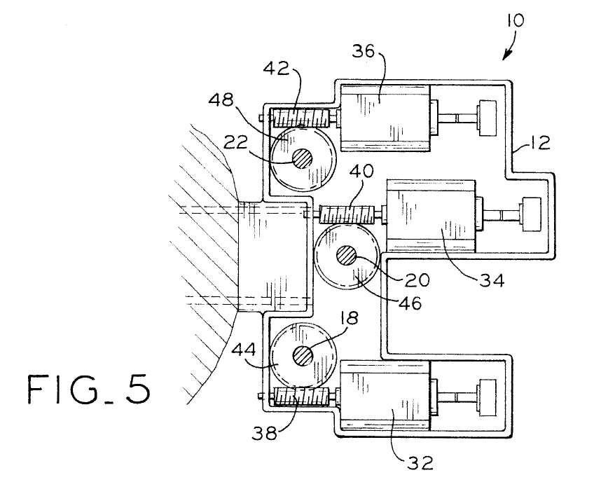

Adjustment arms 18, 20, 22 are received by housing 12 via apertures 26, 28, 30, respectively, extending entirely through housing 12. In one exemplary embodiment, apertures 26, 28, 30 are threaded to threadingly engage adjustment arms 18, 20, 22. As shown in FIG. 5, adjustment arms 18, 20, 22 are operatively connected to variable-speed motors 32, 34, 36, respectively, shown in FIG. 5, and positioned within housing 12. While described and depicted herein with three adjustment arms and three corresponding variable-speed motors, the present invention may be utilized with a single adjustment arm and corresponding motor or, alternatively, any number of adjustment arms and corresponding motors and the present description is not intended to limit the scope of the invention in any manner.

Referring to FIG. 5, adjustment arms 18, 20, 22 are operatively connected to motors 32, 34, 36 by worms 38, 40, 42 and worm gears 44, 46, 48, respectively. Worms 38, 40, 42 and worm gears 44, 46, 48 are meshingly engaged with one another and operate in a known manner to form a worm drive. Specially, worms 38, 40, 42 are rotatable about their own longitudinal axes, but are also translationally fixed to motors 32, 34, 36, respectively. Thus, during operation of motors 32, 34, 36, worms 38, 40, 42 are rotated about their own longitudinal axes, but otherwise remain fixed in longitudinal position.

Worm gears 44, 46, 48 are rotatable about axes transverse to the longitudinal axes of worms 38, 40, 42, but are otherwise fixed in position to substantially prevent longitudinal translation or movements other than rotation of worm gears 44, 46, 48. Thus, during operation of motors 32, 34, 36, worms 38, 40, 42 are rotated along their respective longitudinal axes and the meshing engagement of worm gears 44, 46, 48 therewith results in corresponding rotation of worm gears 44, 46, 48 about axes transverse to the longitudinal axes of worms 38, 40, 42, respectively.

Adjustment arms 18, 20, 22 pass through and are threadingly engaged with worm gears 44, 46, 48, respectively. As result, the rotation of worm gears 44, 46, 48 results in corresponding rotation of adjustment arms 18, 20, 22. As set forth above, adjustment arms 18, 20, 22 are also threadingly engaged with apertures 26, 28, 30 (FIG. 1) of housing 12. Thus, as adjustment arm 18, 20, 22 are rotated by worm gears 44, 46, 48, the engagement of adjustment arms 18, 20, 22 with apertures 26, 28, 30 causes adjustment arms 18, 20, 22 to be threadingly advanced along their respective longitudinal axes through worm gears 44, 46, 48 and apertures 26, 28, 30, respectively.

Additionally, in order to prevent adjustment arms 18, 20, 22 from advancing out of housing 12, adjustment arms 18, 20, 22 include pins 50 extending outwardly therefrom. Pins 50 may be in the form of a solid bar extending through an aperture formed within each of adjustment arms 18, 20, 22 or, alternatively, may be a formed as a projection extending from the exterior surface of adjustment arms 18, 20, 22. Irrespective of how pins 50 are attached, pins 50 extend in a direction substantially transverse to the longitudinal axes of adjustment arms 18, 20, 22 and will contact the lower portion of housing 12 to prevent adjustment arms 18, 20, 22 from advancing out of apertures 26, 28, 30, respectively.

Referring to FIG. 4, by simultaneously operating each of motors 32, 34, 36 in a forward direction, cutter guide 16 will be actuated in the direction of arrow A of FIG. 4 to decrease resection depth D of cutter guide 16. Alternatively, by simultaneously operating each of motors 32, 34, 36 in a reverse direction, cutter guide 16 will be actuated in the direction of arrow B of FIG. 4 to increase resection depth D of cutter guide 16.

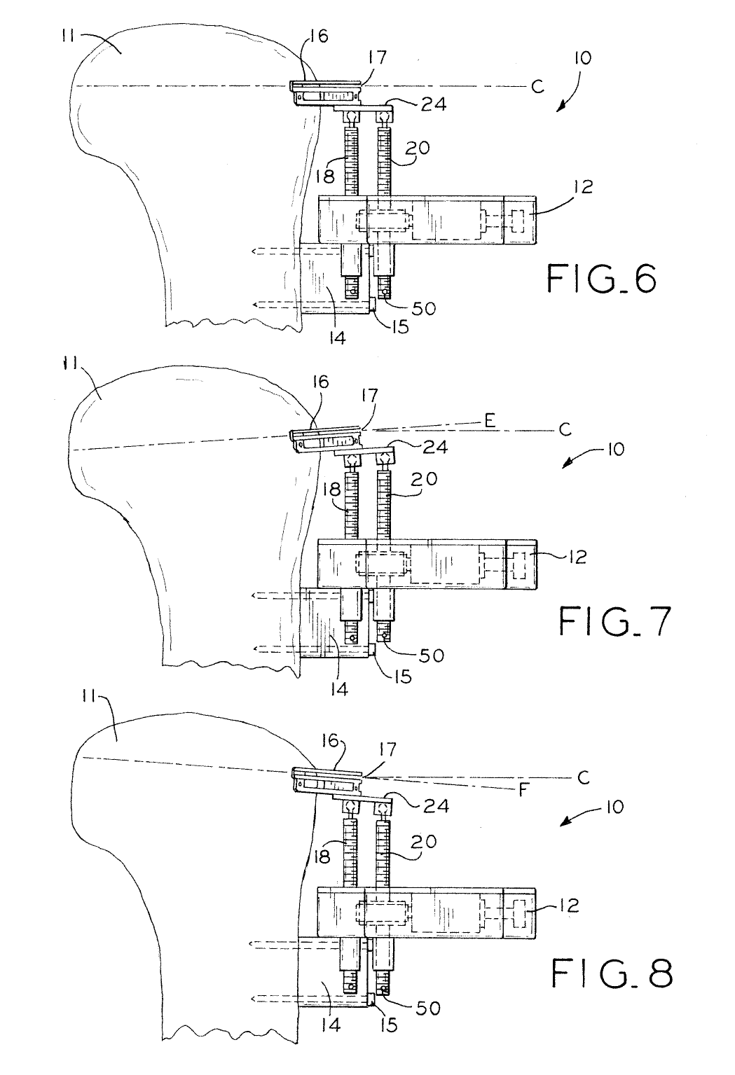

Referring to FIGS. 6-8, cut guide 10 is shown with cutter guide 16 and cut slot 17 positioned at various extension/flexion or anterior/posterior slope angles. Specifically, referring to FIG. 6, cutter guide 16 and cut slot 17 are positioned at a substantially neutral anterior/posterior slope angle, with cut slot 17 substantially aligned with axis C. Referring to FIG. 7, cutter guide 16 and cut slot 17 are positioned at a first, substantially negative anterior/posterior slope angle, with cut slot 17 substantially aligned with axis E. In order to position cutter guide 16 as shown in FIG. 7, adjustment arm 20 is actuated by operation of motor 34. As motor 34 is operated in a forward direction, adjustment arm 20 is moved in the direction of arrow A of FIG. 4, causing the anterior portion of bracket 24 to move in the direction of arrow A and pivot about the ball heads of adjustment arms 18, 22. Alternatively, the negative anterior/posterior slope shown in FIG. 7 could also be achieved by substantially simultaneous operation of motors 32, 36 in a reverse direction. As a result, adjustment arms 18, 22 would move in the direction of arrow B of FIG. 4 and cutter guide 16 would pivot about the ball head of adjustment arm 20.

Referring to FIG. 8, cutter guide 16 and cut slot 17 are positioned at a first, substantially positive anterior/posterior slope angle, with cut slot 17 substantially aligned with axis F of FIG. 8. In order to position cutter guide 16 and cut slot 17 as shown in FIG. 8, motors 32, 36 are substantially simultaneously operated in a forward direction causing adjustment arms 18, 22 to move in the direction of arrow A of FIG. 4. As a result, cutter guide 16 pivots about the ball head of adjustment arm 20. Alternatively, motor 34 may be operated in a reverse direction causing adjustment arm 20 to move in the direction of arrow B of FIG. 4. As a result, cutter guide 16 pivots about the ball heads of adjustment arms 18, 22 to arrive at the position shown in FIG. 8.

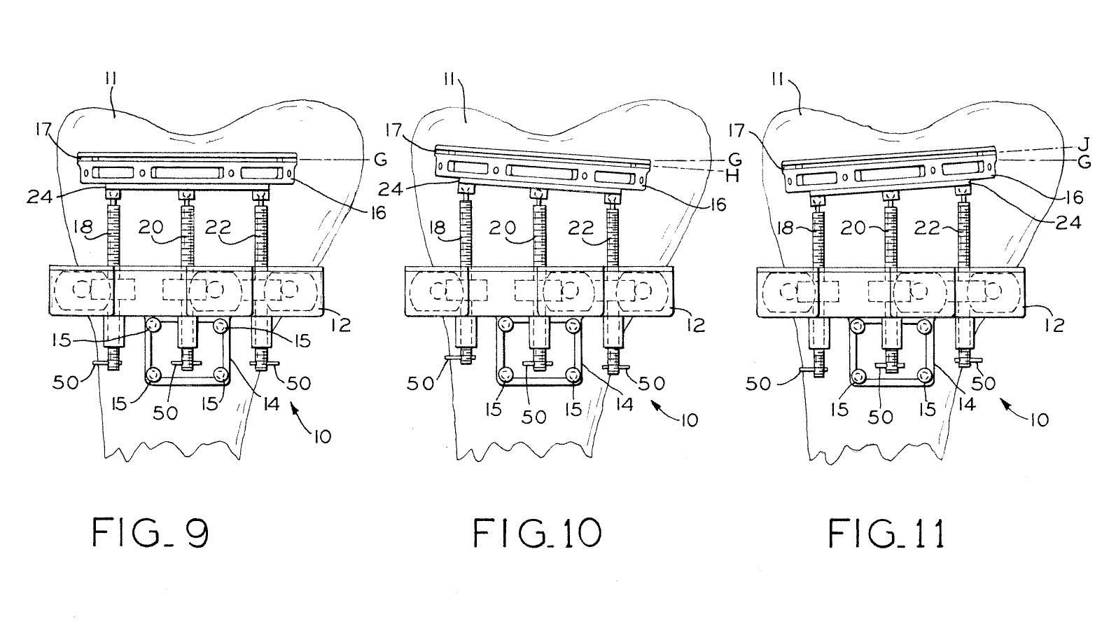

Referring to FIGS. 9-11, cut guide 10 is shown with cutter guide 16 positioned at various varus-valgus angles. Specifically, referring to FIG. 9, cutter guide 16 and cut slot 17 are shown positioned at a substantially neutral varus-valgus angle along axis G. Referring to FIG. 10, cutter guide 16 and cut slot 17 are positioned at a first, substantially negative varus-valgus angle along axis H of FIG. 10. In order to position cutter guide 16 as shown in FIG. 10, motors 32, 34 are operated in a forward direction causing adjustment arms 18, 20 to move in the direction of arrow A of FIG. 4. Specifically, motor 32 is operated at a faster speed than motor 34, causing adjustment arm 18 to move a greater distance in the direction of arrow A than adjustment arm 20. Alternatively, cutter guide 16 and cut slot 17 may be positioned as shown in FIG. 10 by operation of motors 34, 36 in a reverse direction causing movement of adjustment arms 20, 22 in the direction of arrow B of FIG. 4. Specifically, motor 36 is operated at a faster speed than motor 34, causing adjustment arm 22 to move a greater distance in the direction of arrow B than adjustment arm 20.

Referring to FIG. 11, cutter guide 16 and cut slot 17 are positioned at a second, substantially positive varus-valgus angle along line J of FIG. 11. In order to position cutter guide 16 as shown in FIG. 11, motors 34, 36 are operated in a forward direction causing adjustment arms 20, 22 to move in the direction of arrow A of FIG. 4. Specifically, motor 36 is operated at a faster speed than motor 34, causing adjustment arm 22 to move a greater distance in the direction of arrow A than adjustment arm 20. Alternatively, cutter guide 16 and cut slot 17 may be positioned as shown in FIG. 11 by operation of motors 32, 34 in a reverse direction causing movement of adjustment arms 18, 20 in the direction of arrow B of FIG. 4. Specifically, motor 32 is operated at a faster speed than motor 34, causing adjustment arm 18 to move a greater distance in the direction of arrow B than adjustment arm 20.

While described and depict herein with respect to specific movements in single planes, cutter guide 16 may be moved into various positions and along multiple planes, either independently or simultaneously, to achieved a desired position and the descriptions herein are not intended to limit the scope of the invention in any manner.

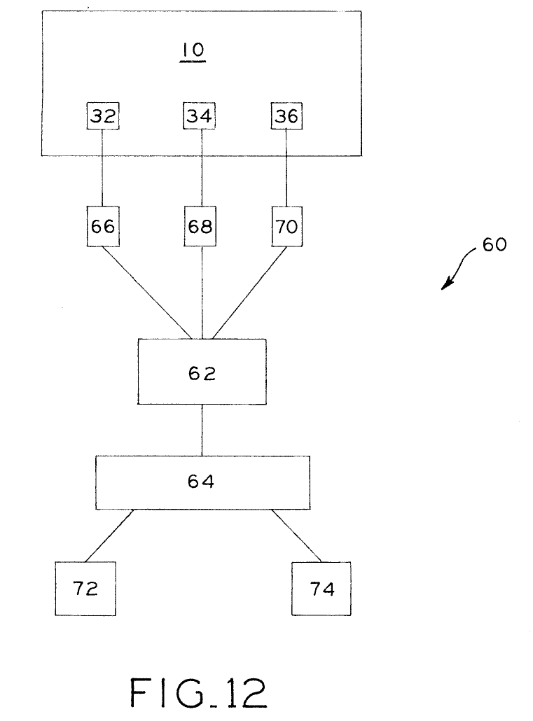

In order to control the movement and position of cutter guide 16, cut guide 10 may be operably connected to cut guide controller 60, shown schematically in FIG. 12. Referring to FIG. 12, cut guide controller 60 includes motor control unit 62, user interface unit 64, and position sensors 66, 68, 70 electronically connected to motors 32, 34, 36, respectively. User interface unit 64 may further include input device 72 and display device 74.

In order to control operation of cut guide 10, a user may input positioning information for cutter guide 16 into user interface unit 64. In one exemplary embodiment, user interface unit 64 is a computer. For example, the user may input positioning information into input device 72. In one exemplary embodiment, input device 72 is a keyboard or other input device configured to operate in conjunction with a computer. The position information inputted by the user may also be displayed by an output device 74. In one exemplary embodiment, output device 74 is a computer monitor or other output device configured to operate in conjunction with a computer, such as a printer.

The positioning information received by user interface unit 64 is then transmitted to motor control unit 62, which is electronically connected to user interface unit 64 and motor motors 32, 34, 36. Once motor control unit 62 receives the positioning information from user interface unit 64, motor control unit 62 calculates the operation of motors 32, 34, 36 necessary to maneuver cutter guide 16 to the position provided by the positioning information. Motor control unit 62 then sends electrical power to motors 32, 34, 36 to activate motors 32, 34, 36.

As described above, motors 32, 34, 36 may be variable-speed motors and, thus, may be actuated to operate at different speeds by motor control unit 62. Additionally, as motors 32, 34, 36 are operated, position sensors 66, 68, 70 track the operation of motors 32, 34, 36, respectively, and provide feedback on the same to motor control unit 62. Based on the feedback received from position sensors 66, 68, 70, motor control unit 62 can calculate the position of adjustment arms 18, 20, 22 and, correspondingly, cutter guide 16. This information may then be sent by motor control unit 62 to user interface unit 64, which may display the information on output device 74. Thus, the user may be visually presented with the position of cut guide 10 in real time. Alternatively, the position of cut guide 10 may be periodically updated to provide the user with the status of the same.

Once adjustment arms 18, 20, 22 are determined by motor control unit 62 to have reached the position inputted by the user, motor control unit 62 ceases operation of motors 32, 34, 36, either simultaneously or independently, as may be necessary. Further, once the desired position is reached, a user may make additional modifications to the position of cutter guide 16 by providing new positioning information to user interface unit 64 in the manner described in detail above, causing the actuation process to be repeated.

While described and depicted herein with specific reference to cut guide controller 60, cut guide 10 may be controlled by any computer assisted surgery system, such as the Navitrack® Navigation System, available from ORTHOsoft, Inc. of Montreal, Canada, and the specific description provided herein is not intended to limit the scope of the invention in any manner.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A cut guide for the resection of a bone, the cut guide comprising:
a housing;
a motor contained within said housing;
a bone mount connected to said housing;
at least one adjustment arm received by said housing and operably connected to said motor, whereby operation of said motor results in movement of said at least one adjustment arm; and
a cutter guide having a cut surface for guiding a cutting tool, said cutter guide attached to said at least one adjustment arm, whereby movement of said at least one adjustment arm results in corresponding movement of said cutter guide.

2. The cut guide of claim 1, wherein said housing further comprises an aperture, said at least one adjustment member received within said aperture.

3. The cut guide of claim 1, wherein said at least one adjustment arm further comprises a threaded pin.

4. The cut guide of claim 3, further comprising a worm gear contained within said housing and operably coupled to said motor and said adjustment arm, wherein movement of said worm gear results in corresponding movement of said adjustment arm.

5. The cut guide of claim 1, further comprising a plurality of adjustment arms attached to said cutter guide and a plurality of motors, each of said plurality of adjustment arms operably connected to a corresponding one of said plurality of motors, whereby the operation of said plurality of motors results in adjustment of at least one of the varus-valgus angle, the extension/flexion angle, and the resection depth of said cutter guide.

6. A cut guide for the resection of a bone, the cut guide comprising:
a housing;
a bone mount connected to said housing;
a cutter guide having a cut surface for guiding a cutting tool; and
means for automatically adjusting the orientation of the guide surface of the cutter guide relative to the housing in response to a user input.

7. The cut guide of claim 6, wherein said means for automatically adjusting the orientation of the guide surface of the cutter guide relative to the housing in response to a user input comprise a user interface and a motor control unit.

8. The cut guide control system of claim 7, wherein said user interface further comprises an input device and a display device.

9. The cut guide control system of claim 7, wherein said user interface unit comprises a computer.

10. The cut guide control system of claim 6, wherein said means for automatically adjusting the orientation of the guide surface of the cutter guide relative to the housing in response to a user input comprises a worm drive.

11. The cut guide control system of claim 10, wherein said worm drive comprises a worm and a worm gear.

12. A method of resecting a bone, comprising:
providing a cutter guide having a guide surface for guiding a cutting tool;
moveably connecting the cutter guide to a bone mount;
mounting the cutter guide to the bone with the bone mount;
providing a user input indicating a desired position of the cutter guide relative to the bone mount and, therefore, the bone; and
automatically actuating the cutter guide to the desired position.

13. The method of claim 12, wherein the step of automatically actuating the cutter guide to the desired position comprises energizing a motor to actuate the cutter guide to the desired position.

14. The method of claim 12, wherein the step of automatically actuating the cutter guide to the desired position comprises energizing a plurality of motors to actuate the cutter guide to the desired position.
